(19) United States
(12) Plant Patent Application Publication
(10) Pub. No.: US 2018/0035594 P1
(43) Pub. Date: Feb. 1, 2018

(54) Phalaenopsis Orchid plant named 'PHALFLAJ'

(50) ;

(71) Applicant: Anthura B.V., Bleiswijk (NL)

(72) Inventor: Martinus Nicolaas Gerardus Van Swieten, Utrecht (NL)

(21) Appl. No.: 15/330,063

(22) Filed: Aug. 1, 2016

(51) Int. Cl. A01H 5/02

(52) U.S. Cl. PLT/311

(57) ABSTRACT

A new and distinct variety of Phalaenopsis plant named ‘PHALFLAJ’, particularly characterized by having small, white flowers, 1 to 4 peduncles that are short and sturdy, leaves that are oblong, and is propagated by meristem tissue culture, is disclosed.

Genus and species: Phalaenopsis hybrid.

Variety denomination: ‘PHALFLAJ’.

### BACKGROUND OF THE NEW PLANT

The present invention relates to a new and distinct cultivar of Phalaenopsis plant, botanically known as Phalaenopsis hybrid of the Orchidaceae family, commonly referred to as moth orchid, and hereinafter referred to by the variety name ‘PHALFLAJ’.

The new Phalaenopsis plant is a product of a planned breeding program conducted by the inventor in Bleiswijk, The Netherlands. The objective of this breeding program was to create a new Phalaenopsis plant with numerous branches and attractive small white flowers, suitable for potted plant production.

The new Phalaenopsis plant ‘PHALFLAJ’ is a result of cross-pollination made by the inventor in October 2005 in Bleiswijk, The Netherlands of the proprietary female, or seed parent, Phalaenopsis hybrid ‘11968-04’ (unpatented) with the proprietary male, or pollen parent, Phalaenopsis hybrid ‘23266-02’ (unpatented).

The new Phalaenopsis was selected by the inventor as a single plant within the progeny of the stated cross-pollination in a controlled greenhouse in Bleiswijk, The Netherlands in June 2009. Asexual reproduction of the new Phalaenopsis plant by meristem tissue culture since March 2012 in Bleiswijk, The Netherlands, has demonstrated that the new variety reproduces true to type with all of the characteristics, as herein described, firmly fixed and retained through successive generations.

Plant Breeder's Rights for this variety have been applied for in Europe on Apr. 18, 2016. ‘PHALFLAJ’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Bleiswijk, The Netherlands and can be used to distinguish ‘PHALFLAJ’ as a new and distinct variety of Phalaenopsis plant.
1) Small, white flowers;
2) 1 to 4 peduncles;
3) Peduncle is short and sturdy; and
4) Shape of the leaf is oblong.

### DESCRIPTION OF THE PHOTOGRAPHS

This new Phalaenopsis plant is illustrated by the accompanying photographs which show the overall plant habit including blooms and foliage of the plant; the colors shown are as true as can be reasonably obtained by conventional photographic procedures. The photographs were taken in a greenhouse in Bleiswijk, The Netherlands, from 50-week old plants in April 2016. Colors in the photographs may differ from the color values cited in the detailed botanical description, which accurately describe the actual colors of the new variety.

FIG. 1 shows the overall plant habit, including blooms and foliage of ‘PHALFLAJ’.

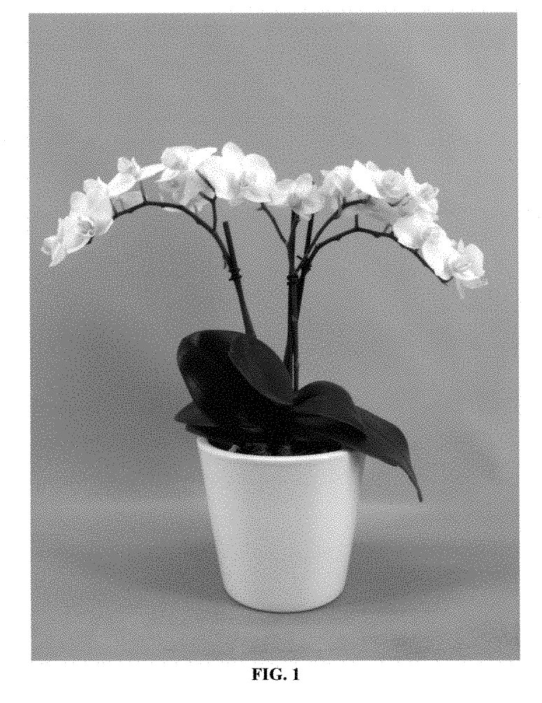

FIG. 2 shows a close-up of a flower of ‘PHALFLAJ’.

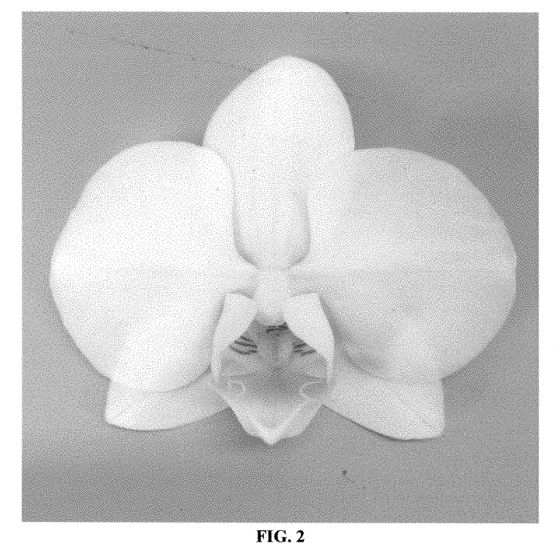

FIG. 3 shows a close-up of the leaves of ‘PHALFLAJ’.

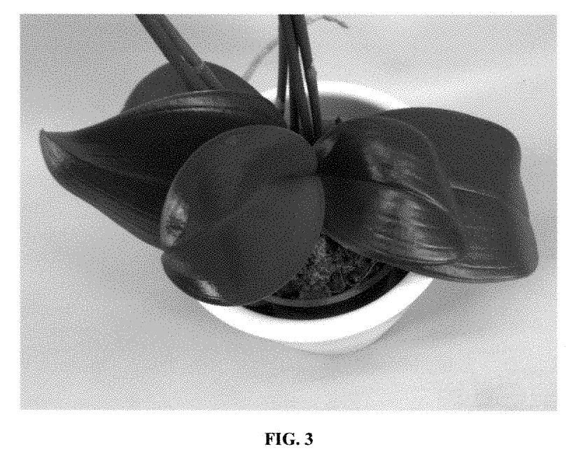

### DESCRIPTION OF THE NEW VARIETY

The following detailed description sets forth the distinctive characteristics of ‘PHALFLAJ’. Plants of the new Phalaenopsis have not been observed under all possible environmental conditions. The phenotype may vary somewhat with variations in environment such as temperature, light intensity and day length, without, however, any variance in genotype. The chart used in the identification of colors described herein is The R.H.S. Colour Chart of The Royal Horticultural Society, London, England, 2015 edition, except where general color terms of ordinary significance are used. The color values were determined under 4000-6000 lux natural light in a greenhouse in Bleiswijk, The Netherlands. Observations and measurements were made in April 2016 on 50-week old plants which were planted from a nursery tray in 12 centimeter (diameter) pots and grown in a greenhouse between 27° C. to 29° C. for 30 weeks, continued by a cooling period of 8 weeks between 18° C. to 20° C. and 12 weeks in a greenhouse of 21° C.

### DETAILED BOTANICAL DESCRIPTION

Classification:
   Family.—Orchidaceae.
   Botanical.—Phalaenopsis hybrid.
   Common name.—Moth orchid.
   Variety name.—‘PHALFLAJ’.
Parentage:
   Female parent.—Phalaenopsis cultivar ‘11968-04’ (unpatented).
   Male parent.—Phalaenopsis cultivar ‘23266-02’ (unpatented).
Propagation:
   Type.—Meristem tissue culture.
Roots:
   Root description.—Greyed-green colored roots with branching lateral roots having light purple and light green colored root tips.
Plant:
   Commercial crop time to flowering.—Approximately 48 to 50 weeks from a rooted cutting to finish in a 12 cm pot.
   Growth habit of peduncle.—Standard, green leaves and panicle.
   Height (from soil level to top of inflorescence).—Approximately 26.0 cm to 36.0 cm.
   Width (measured from leaf tips).—About 17.0 cm to 21.0 cm.
   Vigor.—Strong.
Leaves:
   Mature leaves.—Quantity per plant: 8 to 10 leaves are produced before flowering Length (fully expanded): 12.0 cm to 15.0 cm Width: 8.0 cm to 9.0 cm Shape: Oblong Base shape: Slightly elongated Apex: Rounded unequal Leaf blade angle with the petiole (measured from the horizontal position): Between 0 degrees and 25 degrees Leaf margin: Entire Color: Upper surface: RHS NN137B Lower surface: RHS 146A Texture: Smooth Thickness: 2.0 mm to 2.3 mm Venation: Pattern: Parallel Color of the midvein: Upper surface: RHS 147A Lower surface: RHS 146A.
Peduncle:
   Quantity per plant.—1 to 4.
   Number of flowers per peduncle.—14 to 23.
   Length.—26.0 cm to 36.0 cm.
   Diameter.—4.1 mm to 4.3 mm.
   Strength.—Strong.
   Aspect.—Upright.
   Texture.—Smooth.
   Color.—Green (RHS 146A) with a touch of dark purple (RHS 187A).
   Internode length.—3.5 cm to 4.5 cm.
   Callosities.—None.
Inflorescence description:
   Appearance.—Upright to slightly pendant, panicle inflorescence with bilaterally symmetrical flowers that open in succession beginning with the lowermost flower.
   Inflorescence size.—Height (from base to tip): 120.0 mm to 140.0 mm.
   Flowering time.—First flowers can be expected 10 to 11 months after planting in a 12 cm (diameter) pot.
   Flower.—Height: 59.0 mm to 61.0 mm Diameter: 62.0 mm to 64.0 mm Depth of lip: 15.0 mm to 17.0 mm.
   Flower longevity.—On the plant: 5 to 8 weeks. Fragrance: Absent.
   Flower bud.—Average size: Medium Length: 16.0 mm to 18.0 mm Width: 13.0 mm to 15.0 mm Shape: Egg shaped Color: Green (RHS N144D).
   Petals.—Arrangement: Open/free Shape: Semi-circular Apex: Rounded asymmetric Margin: Entire Length (from base to tip): 28.0 mm to 30.0 mm Width: 34.0 mm to 36.0 mm Color (when fully opened): Upper surface: Basic color: White (RHS NN155C) Over color: Absent Lower surface: Basic color: White (RHS NN155C) Over color: Absent.
   Dorsal sepal.—Shape: Elliptic Apex: Obtuse symmetric Margin: Entire Length (from base to tip): 31.0 mm to 33.0 mm Width: 22.0 mm to 24.0 mm Color (when fully opened): Upper surface: Basic color: White (RHS NN155C) Over color: Absent Lower surface: Basic color: White (RHS NN155C) Over color: Absent.
   Lateral sepals.—Shape: Ovate Apex: Obtuse asymmetric Margin: Entire Length (from base to tip): 31.0 mm to 33.0 mm Width: 18.0 mm to 20.0 mm Color (when fully opened): Upper surface: Basic color: White (RHS NN155C) Over color: Absent Lower surface: Basic color: White (RHS NN155C) Over color: Light green (RHS 145D).
   Labellum (lip).—Margin: Entire Whiskers: Present Length of whiskers: 12.0 mm to 14.0 mm Color of whiskers: White (RHS NN155C) Pubescence on the lip: Absent.
   Lateral lobe.—Shape: Spatulate Length (from base to tip): 16.0 mm to 18.0 mm Width: 10.0 mm to 12.0 mm Color: White (RHS NN155C) and slightly green (RHS 150B) on one side with a few purple (RHS 187C) stripes towards the base.
   Apical lobe.—Shape: Triangular Length: 14.0 mm to 16.0 mm Width: 17.0 mm to 19.0 mm Color: White (RHS NN155C) with green (RHS 150B) towards the callus.
   Callus.—Average size: Small to medium Height: 0.5 cm to 0.7 cm Length: 0.3 cm to 0.5 cm Width: 0.2 cm to 0.4 cm Color: Yellow (RHS 12B) and dotted.
   Reproductive organs.—Column: Length: 7.0 mm to 9.0 mm Diameter: 4.0 mm to 4.2 mm Color: White (RHS NN155C) Pollinia: Quantity: 2 Diameter: 0.9 mm to 1.1 mm Color: Yellow-orange (RHS 23A) Ovary: Length: 7.0 mm to 9.0 mm Diameter: 2.0 mm to 2.2 mm Pedicel: Length: 29.0 mm to 31.0 mm Diameter: 2.2 mm to 2.5 mm Color: Green (RHS NN137B) and light green (RHS 144D) towards the flower.
Disease, pest, and stress resistance: No specific resistance or susceptibility observed to pathogens and pests common to Phalaenopsis.
Fruit and seeds: Fruit and seed development has not been observed on plants of the new Phalaenopsis.

### COMPARISON WITH PARENTAL AND COMMERCIAL VARIETIES

‘PHALFLAJ’ differs from female parent plant ‘11968-04’ (unpatented) in that ‘PHALFLAJ’ has a small to medium callus, darker green peduncles with a touch of dark purple, a rounded petal apex, and a striped lateral lobe, whereas ‘11968-04’ has a medium callus, lighter green peduncles, an obtuse petal apex, and a striped and dotted lateral lobe. Additionally, ‘PHALFLAJ’ has longer petals and whiskers than ‘11968-04’.

The male parent of ‘PHALFLAJ’, cultivar ‘23266-02’ (unpatented), is no longer in existence, so a meaningful comparison cannot be made.

‘PHALFLAJ’ is most similar to the commercial Phalaenopsis plants named ‘PHALBAPAK’ (U.S. Plant Pat. No. 27,508) and ‘PHALDUXI’ (U.S. Plant Pat. No. 25,680). ‘PHALFLAJ’ differs from the commercial variety ‘PHALBAPAK’ in that ‘PHALFLAJ’ has lateral sepals with no over color and dorsal sepal lower surfaces with no over color, whereas ‘PHALBAPAK’ has lateral sepals with light green and light purple over color towards the apex, and dorsal sepal lower surfaces with light purple and yellow-green over color. Additionally, ‘PHALFLAJ’ has shorter petals than ‘PHALBAPAK’.

‘PHALFLAJ’ differs from the commercial variety ‘PHALDUXI’ in that ‘PHALFLAJ’ has lateral sepals with even coloring at the base and a rounded petal apex, whereas ‘PHALDUXI’ has lateral sepals with dots/spots at the base and a mucronate petal apex. Additionally, ‘PHALFLAJ’ has longer petals than ‘PHALDUXI’.

I claim:

1. A new and distinct variety of Phalaenopsis plant named ‘PHALFLAJ’, substantially as described and illustrated herein.
